(19) United States
(12) Patent Application Publication
(10) Pub. No.: US 2006/0004438 A1
(43) Pub. Date: Jan. 5, 2006

(54) Prosthesis, delivery system and method for neurovascular aneurysm repair

(75) Inventors: Miles Alexander, Sunnyvale, CA (US); Eric Leopold, Redwood City, CA (US); David Chi, Corona Del Mar, CA (US)

Correspondence Address: LUCE, FORWARD, HAMILTON & SCRIPPS LLP, 11988 EL CAMINO REAL, SUITE 200, SAN DIEGO, CA, 92130, US

(73) Assignee: Novostent Corporation, Mountain View, CA (US)

(21) Appl. No.: 11/154,056

(22) Filed: Jun. 15, 2005

Related U.S. Application Data
(63) Continuation-in-part of application No. 10/836,909, filed on Apr. 30, 2004.

(51) Int. Cl. A61F 2/06

(52) U.S. Cl. 623/1.22

(57) ABSTRACT

The present invention is directed to a prosthesis for treating an aneurysm, and delivery systems and methods therefor. The prosthesis includes a radially expanding distal section coupled to a helical section, the helical section including a localized feature configured to exclude or retard blood flow into an aneurysm. Methods of loading the prosthesis onto a specially-designed delivery system that facilitates proper orientation of the prosthesis within a target vessel, and methods of using the delivery system to deliver the prosthesis, also are provided.

### CROSS REFERENCE TO RELATED APPLICATIONS

This application is a continuation-in-part of U.S. patent application Ser. No. 10/836,909, filed Apr. 30, 2004, and entitled “DELIVERY CATHETER THAT CONTROLS FORESHORTENING OF RIBBON-TYPE PROSTHESES AND METHODS OF MAKING AND USE”.

### FIELD OF THE INVENTION

The present invention relates to prostheses and methods for treating aneurysms in very small vessels, such as the cerebral vessels. More particularly, the present invention is directed to the use of helically wound stent including one or more features for retarding or excluding blood flow into an aneurysm sac.

### BACKGROUND OF THE INVENTION

Today there are a wide range of intravascular prostheses on the market for use in the treatment of aneurysms, stenosis, and other vascular irregularities. Balloon expandable and self-expanding stents are well known for restoring patency in a stenosed vessel, e.g., after an angioplasty procedure, and the use of coils and stents are known techniques for treating aneurysms.

Previously-known self-expanding stents generally are retained in a contracted delivery configuration using a sheath, then self-expand when the sheath is retracted. Such stents commonly have several drawbacks, for example, the stents may experience large length changes during expansion (referred to as “foreshortening”) and may shift within the vessel prior to engaging the vessel wall, resulting in improper placement. Additionally, many self-expanding stents have relatively large delivery profiles because the configuration of their struts limits further compression of the stent. Accordingly, such stents may not be suitable for use in smaller vessels, such as cerebral vessels and coronary arteries.

Other drawbacks associated with the use of coils or stents in the treatment of aneurysms is that the devices, when deployed, may have a tendency to straighten or otherwise remodel a delicate cerebral vessel, which may cause further adverse consequences. Moreover, such devices may not adequately reduce or exclude blood flow from the vessel into the sac of the aneurysm, and thus may not significantly reduce the risk of rupture.

For example, U.S. Pat. No. 6,660,032 to Klumb et al. describes a stent comprising a pair of helical mesh coils interconnected by ladder-like cross members and entirely covered by a graft material. In operation, the stent may be wound into plurality of turns of reduced diameter, and then constrained within a delivery sheath. The delivery sheath is retracted to expose the distal section of the stent and anchor the distal end of the stent. As the delivery sheath is further retracted, subsequent individual turns of the stent unwind to conform to the diameter of the vessel wall.

The stent described in the foregoing publication has several drawbacks. For example, the use of graft material along the full length of the stent increases the overall delivery profile of the stent, potentially rendering the device too large and too axially stiff for use in treating aneurysms located in narrow or tortuous neurovascular vessels. In addition, the presence of graft material along the full length of the stent may cause inadvertent closure of perforators—small side vessels. Moreover, due to friction between the turns and the sheath, the individual turns of the stent may bunch up, or overlap atop one another, when the delivery sheath is retracted. This in turn may create gaps in the stent that inadequately limit the flow of blood from the vessel into the sac of an aneurysm.

U.S. Pat. No. 4,768,507 to Fischell et al. and U.S. Pat. No. 6,576,006 to Limon et al., each describe the use of a groove disposed on an outer surface of an interior portion of the stent delivery catheter, wherein at least a portion of the stent is disposed within the groove to prevent axial movement during proximal retraction of the sheath. While the delivery catheters disclosed in these patents may reduce axial movement and bunching of the prosthesis during retraction of the sheath of the delivery catheter, those systems do not effectively address the issue of stent foreshortening nor eliminate the creation of gaps that permit blood to circulate into the sac of an aneurysm. For example, once the sheath of the delivery catheter is fully retracted, the turns of the stent may shift relative to one another within the vessel prior to engaging the vessel wall, resulting in inadequate coverage of the stenosis or aneurysm.

Aneurysms often arise in smaller vessels at bends, where a change in the direction of blood flow results in high hemodynamic loads being exerted on the vessel wall. Aneurysms thus are often encountered at bifurcations and on the outer bends of tortuous vessels, where flow impinges on the vessel wall and is redirected. Aneurysm repair typically requires surgical intervention, although some efforts to develop percutaneous solutions have been made.

One previously-known method of treating aneurysms percutaneously involves deploying platinum coils within the aneurysm sac, thereby causing the blood contained within the sac to clot. In such cases, a microcatheter may be disposed with its tip extending into the aneurysm sac. One or more embolization coils are ejected from the tip of the microcatheter into the sac, precipitating clotting of the blood contained within the aneurysm sac. During the clotting process it is possible for thrombus to enter blood flowing past or through the aneurysm, thereby creating a risk of blocking downstream vessels.

In view of the above-identified drawbacks of previously-known methods for percutaneously treating aneurysms of small vessels, it would be desirable to provide prostheses and methods for treating aneurysms that substantially retard or exclude flow into an aneurysm sac.

It also would be desirable to provide prostheses and methods for use in treating aneurysms of small or tortuous vessels, wherein prostheses have a small delivery profile that facilitates passage through narrow vessels.

It further would be desirable to provide prostheses for use in treating aneurysms of small or tortuous vessels, wherein the prostheses have a high degree of axial flexibility thereby further facilitating delivery through tortuous vessels.

### SUMMARY OF THE INVENTION

In view of the foregoing, it is an object of the present invention to provide percutaneously-deliverable prostheses and methods for treating aneurysms of small vessels, wherein the prostheses substantially retard or exclude flow into an aneurysm sac.

It is another object of the present invention to provide prostheses and methods for use in treating aneurysms of small or tortuous vessels, wherein the prostheses have a small delivery profile that facilitates delivery through narrow vessels.

It is a further object of this invention to provide prostheses and methods for use in treating aneurysms of small or tortuous vessels, wherein the prostheses have a high degree of axial flexibility, thereby enabling delivery through tortuous vessels.

These and other objects of the present invention are accomplished by providing a prosthesis, delivery system and methods wherein the prosthesis includes a self-expanding helical section including a localized feature that retards or excludes blood flow into the sac of an aneurysm. The feature may comprise a segment of graft material disposed only for a discrete portion of the circumference of the prosthesis or a local variation in the pattern of struts making up the prosthesis.

In a preferred embodiment, the prosthesis comprises a radially self-expanding distal section coupled to a helically-wound proximal section, wherein the proximal section has a localized feature configured to retard or exclude blood flow into an aneurysm. The feature may comprise an area on the helical section having a locally higher material concentration designed to span the neck of the aneurysm, or graft material disposed on the helical section for a predetermined axial length. Compared to previously known prosthesis designs, such as described in the foregoing patent to Klumb et al., the localized nature of the aneurysm exclusion feature is expected to provide a prosthesis that can be wound to a substantially smaller delivery profile while retaining a high degree of axial flexibility.

In accordance with another aspect of the present invention, a specially configured delivery system is provided for use with the inventive prosthesis to assist the clinician in orienting and delivering the prosthesis within a target vessel. The delivery system preferably comprises a catheter having a predetermined non-circular cross-section that cooperates with the tortuosity of the patient's anatomy to facilitate proper angular orientation of the vascular prosthesis within the vessel. For example, the delivery catheter may comprise a substantially elliptical profile that automatically orients the catheter within the vessel with a known orientation.

In accordance with a further aspect of this invention, a method of marking a desired deployed location of a localized feature on the helical section of the prosthesis is provided. The method includes the steps of selecting a reference point on the delivery catheter, determining the axial location of the reference point, determining the axial and angular location of the feature and providing a reference mark on the prosthesis to indicate the desired deployed location of the feature.

Methods of using the prosthesis and delivery system of the present invention for treating aneurysms in small vessels, such as the cerebral vessels, also are provided.

### BRIEF DESCRIPTION OF THE DRAWINGS

Further features of the invention, its nature and various advantages will be more apparent from the accompanying drawings and the following detailed description of the preferred embodiments, in which:

FIG. 1 is a perspective view of a prosthesis constructed in accordance with the principles of the present invention;

FIG. 2 is a perspective view of an alternative embodiment of a prosthesis of the present invention;

FIGS. 3A and 3B are, respectively, side view and cross-sectional views of a delivery system of the present invention;

FIGS. 4A and 4B are, respectively, side and end views depicting the location of a therapeutic feature in accordance with the principles of the present invention;

FIG. 5 is a side view of the prosthesis of FIGS. 4, wherein the helical section has been flattened;

FIG. 6 is a side view of the vascular prosthesis of FIG. 4A disposed around a distal end of the delivery catheter of FIGS. 3;

FIGS. 7A and 7B are side and cross-sectional views, respectively, of the vascular prosthesis and delivery catheter of FIGS. 3, wherein the vascular prosthesis is in the deployed configuration; and

FIG. 8 is a cross-sectional views showing a method of deploying the prosthesis of the present invention using the delivery system of FIGS. 3.

### DETAILED DESCRIPTION OF THE INVENTION

The present invention is directed to prostheses, delivery systems and methods for treating aneurysms located within narrow and tortuous vessels, such as in the cerebral vasculature. In accordance with the principles of the present invention, the prosthesis includes a feature disposed on a localized region of the prosthesis to retard or exclude blood flow into the sac of an aneurysm. The prosthesis may be used alone or in conjunction with embolism coils, such as are known in the art.

In accordance with the principles of the present invention, the aneurysm exclusion feature comprises a locally-higher density of the strut arrangement of the prosthesis or a portion of graft material disposed only on a discrete portion of the length or circumference of the prosthesis. Due to the localized nature of the feature, the prosthesis of the present invention is expected to provide a smaller delivery profile, and greater flexibility and trackability than previously-known devices.

Further in accordance with the invention, a delivery system is provided that facilitates deployment of the prosthesis in the vessel with a specified angular and axial alignment. The delivery catheter also provides a predictable degree of foreshortening of the stent, including substantially zero foreshortening. The catheter also preferably includes a radio-opaque marker arrangement and non-circular cross-section that facilitate delivery of the prosthesis with a desired orientation with a target vessel.

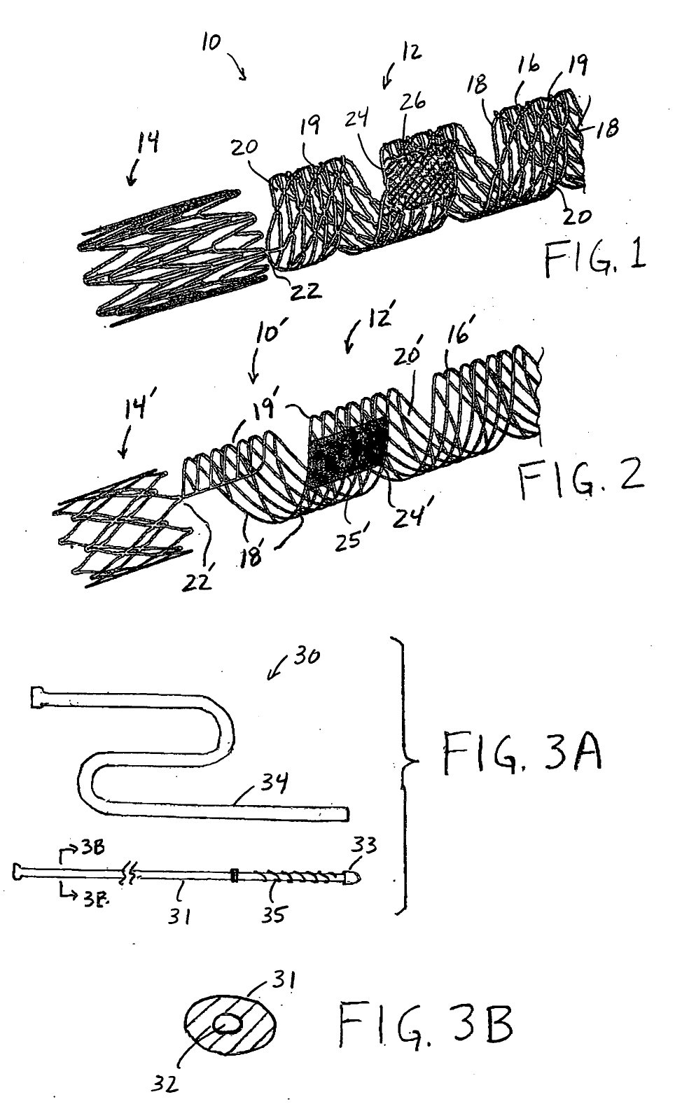

Referring to FIG. 1, a preferred vascular prosthesis of the present invention is described. As used in this specification, the terms “vascular prosthesis” and “stent” are used interchangeably. Vascular prosthesis 10 is described in copending commonly assigned U.S. patent application Ser. No. 10/342,427, filed Jan. 13, 2003, and comprises helical section 12 and distal section 14, each capable of assuming contracted and deployed states. In FIG. 1, helical section 12 and distal section 14 each are depicted in their respective deployed states.

Vascular prosthesis 10 preferably is formed from a solid tubular member comprising a shape memory material, such as nickel-titanium alloy (commonly known as “Nitinol”), using laser cutting techniques that are per se known in the art. The prosthesis is then subjected to an appropriate heat treatment, also known in the art, while the device is held in the desired deployed configuration (e.g., on a mandrel), thus conferring a desired deployed configuration to vascular prosthesis 10 when self-deployed.

Distal section 14 is configured to expand radially outward from its contracted position, and comprises a pattern of cells, illustratively having a zig-zag or diamond configuration in the deployed state. Distal section 14 is designed to be deployed from a delivery catheter first to fix the distal end of the stent at a desired location within a vessel. In this manner, subsequent deployment of helical section 12 of the stent may be accomplished with greater accuracy.

Helical section 12 comprises mesh 16 having a selected cell pattern formed by multiplicity of struts 18, wherein the mesh defines a plurality of substantially flat turns 19. Struts 18 further define a multiplicity of openings 20. Turns 19 are configured to be wound down onto a delivery system in the contracted delivery configuration, as described in greater detail below, in an overlapping manner. It should be understood that the configuration of helical section 12 depicted in FIG. 1 is merely illustrative, and other patterns may be advantageously employed. Helical section 12 is coupled to distal section 14 at junction 22.

Still referring to FIG. 1, in accordance with one aspect of the present invention, helical section 12 further includes localized feature 24 configured to exclude or reduce flow into an aneurysm sac. Feature 24 may comprise a locally denser arrangement of struts 26, as depicted in FIG. 1, configured to impede blood flow into the sac of an aneurysm. These struts also may be configured to allow for separation or deflection, for example, so that a microcatheter may pass between the struts to deliver coagulation coils. Feature 24 may extend for the several consecutive turns 19, or only for part of the circumference of a single turn. As will be appreciated by one of skill in the art of helical stent design, the higher the concentration of struts 26 in feature 24, the greater the axial rigidity of the prosthesis at that axial location. Accordingly, it is desirable to make the length of feature 24 as short as possible to retain axial flexibility and trackability of the stent. In addition, by providing feature 24 on only as much of helical section 12 as required for a particular application, the overall delivery profile of the prosthesis may be kept substantially smaller than previously-known stent designs.

Referring now to FIG. 2, an alternative embodiment of the vascular prothesis of the present invention is described, in which like parts are identified with like-primed numbers to those used in FIG. 1 (e.g., prosthesis 10′). Prosthesis 10′ includes helical section 12′ and distal section 14′. As in the embodiment of FIG. 1, distal section 14′ is configured to expand radially outward from its contracted position, and comprises a pattern of cells, illustratively having a zig-zag or diamond configuration in the deployed state.

Helical section 12′ comprises mesh 16′ having a selected cell pattern formed by multiplicity of struts 18′ to form plurality of turns 19′. Struts 18′ define multiplicity of openings 20′. Helical section 12′ is coupled to distal section 14′ at junction 22′ and further includes localized feature 24′ configured to exclude or reduce flow into an aneurysm sac. Feature 24′ comprises a portion of graft material 25′, for example, such as expanded PTFE or polyurethane, glued or sintered onto struts 18′ for a predetermine number of turns 19′ or only for part of the circumference of a single turn.

Polyurethane, for example, would provide a thin wall that could be readily pierced by a microcatheter to deliver coils, and would substantially self-seal once the microcather was removed. By providing feature 24′ on only as much of helical section 12′ as required for a particular application, the overall delivery profile of the prosthesis may be kept substantially smaller than previously-known stent designs, such as the Klumb et al. patent mentioned above.

Referring now to FIGS. 3, delivery catheter 30 of the present invention is described. Delivery catheter 30 includes inner member 31 having central lumen 32, distal tip 33 and sheath 34. Sheath 34 may comprise a polymeric material disposed on a metal braiding and having good flexibility and sufficient radial strength to retain a stent in the contracted delivery configuration on inner member. Sheath 34 may comprise, for example, a stainless steel braid covered by polyurethane or polyethylene material and preferably includes a lubricious inner surface to facilitate retraction of the sheath during stent delivery.

Inner member 31 is constructed so as to mitigate or eliminate foreshortening during deployment by imposing on the stent in the contracted delivery configuration the same wrap angle e that the stent will have in the deployed configuration. This is accomplished by forming helical ledge 35 on the outer surface of inner member 31. Ledge 35 may be formed in a number of ways, such as by gluing, soldering or laminating a helical wire to the outer surface of the inner member, by braiding a helical wire into fibers forming the inner member, or by integrally forming the ledge with the inner member, e.g., using an extrusion or molding process.

During wrapping of a stent onto inner member 31, either a proximal or distal edge of the stent is abutted against helical ledge 35, so that adjacent turns of the stent overlap one another. Helical ledge 35 also provides linear resistance to stent migration when sheath 34 is retracted during stent deployment. This engagement between the turns of the stent and the inner member maintains the linear stability of the stent, and reduces the risk that overlapping turns of the stent bunch up or seize against the interior surface of the sheath. Moreover, the helical ledge ensures that the stent unwinds on its axis but does not experience significant linear change along the axis. Further details regarding the construction of inner member 31 are provided in co-pending, commonly assigned U.S. patent application Ser. No. 10/836,909, filed Apr. 30, 2004, the entirety of which is incorporated herein by reference.

Applicants have observed that aneurysms frequently occur on the outer bends of the smaller vessels due to the hemodynamic loading on the vessel wall associated with redirecting blood flow. Thus, for example, aneurysms frequently occur near bifurcations. In accordance with the foregoing observation, applicants have designed inner member 31 to have a non-circular, and preferably elliptical, cross-section, as shown in FIG. 3B. Due to the elliptical shape of the inner member, delivery catheter 30 will pass through tortuous anatomy with a known orientation. More particularly, the delivery catheter will automatically orient itself within a vessel so that the major axis of the ellipse faces the outside of the curve.

As described in greater detail below, feature 24 or 24′ of the prosthesis 10 or 10′ may be loaded with a predetermined orientation into the delivery catheter relative to the circumference of inner member 31. Then, when the delivery catheter and stent are advanced into the target vessel, the non-circular shape of the delivery catheter will ensure that stent is oriented with the vessel so that the feature spans the aneurysm. Embolization coils then may be delivered into the aneurysm sac or attached to the prosthesis to treat the aneurysm.

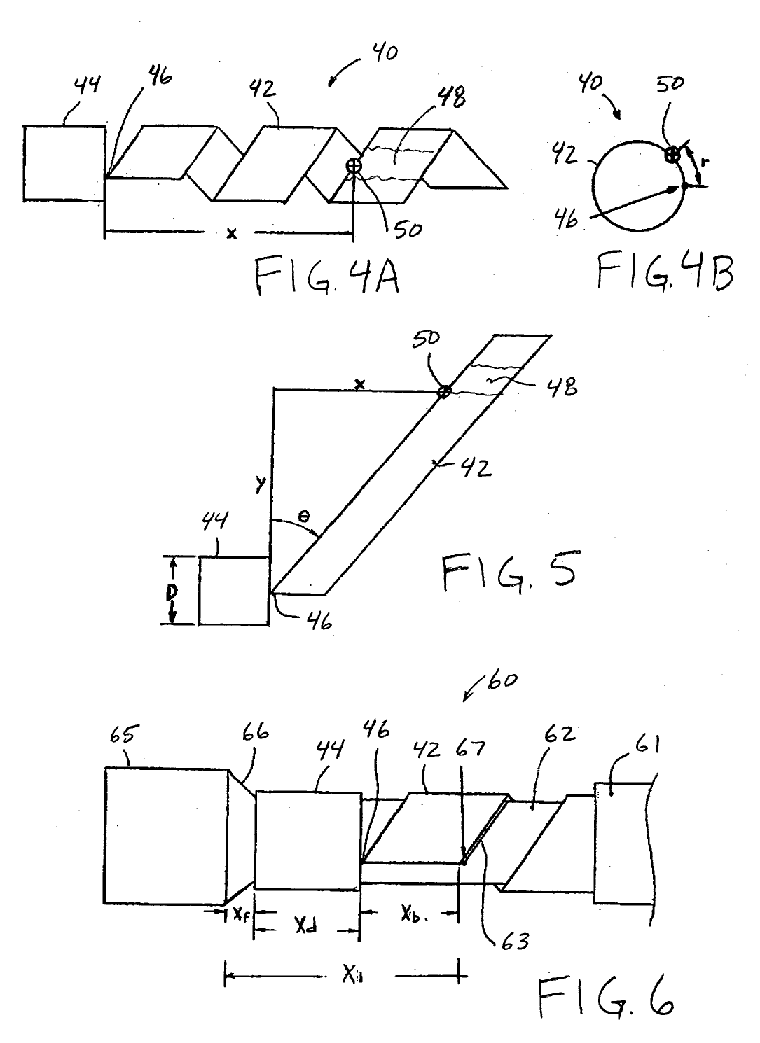

Referring now to FIGS. 4A and 4B, a method of positioning the vascular prosthesis within the delivery system of FIGS. 3 is described. In order to properly place the vascular prosthesis at a desired location within a vessel with feature 24 or 24′ in apposition to the aneurysm neck, it is necessary to accurately determine the location of the feature both axially and circumferentially relative to helical section 12 or 12′. This in turn requires determination of the relationship of the feature location between the expanded deployed configuration and the contracted delivery configuration.

Referring to FIGS. 4A and 4B, vascular prosthesis 40 comprises helical section 42 coupled to distal section 44 at junction 46. Prosthesis 40 includes feature 48, such as locally higher strut density or graft material, configured to exclude or reduce blood flow into an aneurysm. Radio-opaque mark 50 is disposed on helical section 42 to indicate the location of the distal edge of feature 48 on vascular prosthesis 40. Feature 48 is defined by two variables: axial distance (x) from the junction 46 and angular distance (r). Junction 46 defines a reference point wherein xⱼ=0 and rⱼ=0.

When designing a vascular prosthesis having feature 48 in accordance with the present invention, axial distance x is pre-defined. By way of example, consider a vascular prosthesis design that requires a feature three-quarters of the distance from the distal end of the helical body. Once axial distance x is defined, the angular location may be calculated using the equations set forth in the next paragraph.

Referring to FIG. 5, vascular prosthesis 40 is shown with helical section 42 flattened out for illustrative purposes. Mark 50 is disposed on helical section 42 to indicate the location of the distal edge of the feature, defined by axial distance x and angular distance r. Angle (θ) and diameter (D₂) of the deployed helical body preferably are determined by design. Axial distance x of the vascular prosthesis feature also is known, whereas circumferential distance (y) of the feature is determined as y=Π*D (for exactly one revolution) or y=Π*D*r/360 (for a partial or more than one revolution). Using the known formula for a tangential relationship (tan(θ)=x/y), r is solved in terms of x: tan(θ)=x/y=x/(Π*D*r/360). By solving for angular location r, the following equation is obtained: r=360*x/Π*D*tan(θ).

When a feature is present after the first revolution (i.e., r>360), then the number of revolutions to the feature is determined by r/360, thereby resulting in a fractional number. When a feature is disposed at the same angular location as the junction 46, then r/360 is an integer. Otherwise, there is a fractional portion that is equal to the angular change relative to the last full revolution. By way of example, if r/360=3.25, there are 3 full revolutions and an additional one-quarter revolution (90°) past the angular location of the junction.

The relationship between changes in diameter D and changes in angular location r must be determined to accurately wrap the prosthesis onto the delivery catheter for deployment in different size vessels. For a helix, axial distance x does not change (x₁=x₂) when diameter D changes from D₁ to D₂, as long as angle θ remains constant (θ₁=θ₂).

Using the equation r=360*x/Π*D*tan(θ), axial distance x is solved for: x=Π*D*r*tan(θ)/360. Because axial distance x₁ equals axial distance x₂: Π*D₁*r₁*tan(θ)/360=Π*D₂*r₂*tan(θ)/360. Solving for r₂, the following equation is obtained: r₂=D₁*r₁/D₂. Using this equation, the angular location of one or more features on the vascular prosthesis may be determined at different diameters. In general, angular location r changes proportionally with changes in diameter D.

FIG. 6 depicts the distal end of delivery catheter 60 constructed as described above with respect to FIG. 3A. Delivery catheter 60 includes retractable sheath 61 and inner member 62 having helical ledge 63 disposed thereon. Delivery catheter 60 further comprises distal marker 65 attached to inner member 62 via fillet 66. During wrapping of a vascular prosthesis onto inner member 62, the distal turn of helical body 42 is abutted against helical ledge 63, which acts as a guide for wrapping subsequent turns around the inner member. In the illustrated embodiment, adjacent turns of the stent do not overlap one another in the delivery configuration.

Still referring to FIG. 6, a method of marking the expected location of a feature on the vascular prosthesis is described. Initially, a reference point on the delivery catheter is selected. Illustratively, the longitudinal edge of the distal turn of the prosthesis is aligned with distal end 67 of helical ledge 63, which is used as a reference point. Of course, other locations may be selected as the reference point without departing from the scope of the invention.

Starting at the proximal edge of distal marker 65, the axial location (x₁) of the distal end of helical ledge 63 is determined by adding: (1) the axial length of the fillet (x_{f}); (2) the axial length of the distal section (x_{d}); and (3) the axial length of one turn of the helical body (x_{b}). Thus, the following equation is obtained for the axial location of the distal end of the helical ledge: x₁=x_{f}+x_{d}+x_{b}. If junction 46 is aligned with distal end 67 of helical ledge 63, then rⱼ=r₁=0. The axial and angular location of the feature now may be calculated using axial location x₁ as the reference point.

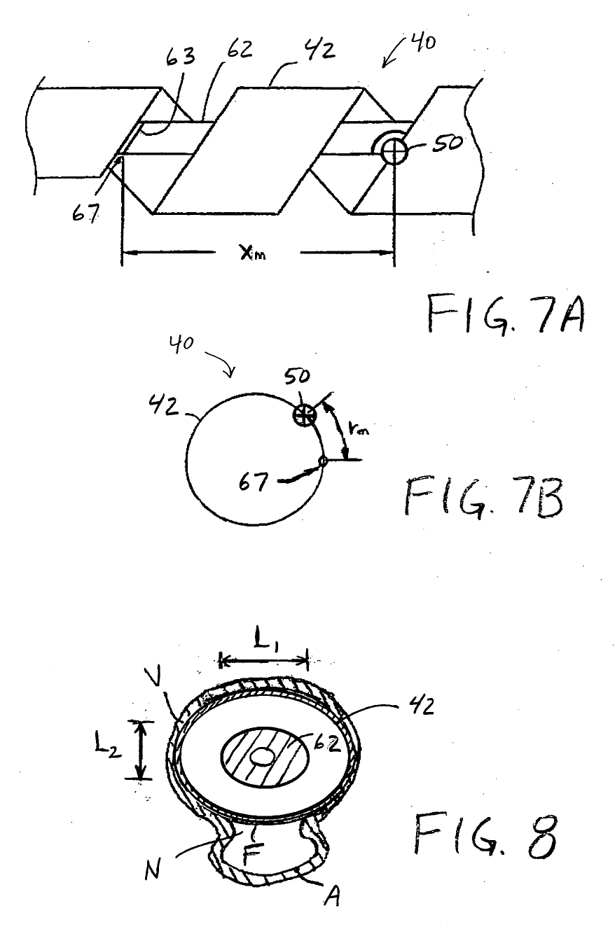

Referring to FIGS. 7A and 7B, the helical section 42 of prosthesis 40 is shown in the expanded deployed configuration prior to withdrawal of inner member 62 of delivery catheter 60. Mark 50 is disposed on helical body 42 so that its axial location is defined by (xₘ) and its angular location is defined by (rₘ). The location of mark 50 is related to deployed diameter (D_{dep}, x_{dep}, r_{dep}), such that: (1) xₘ=x_{dep}−x_{b}; and (2) rₘ=r_{dep}=360*x_{dep}/Π* D_{dep}*tan(θ). Using these two equations, the delivery catheter is configured to include a mark that indicates the axial and angular location of a feature on the vascular prosthesis.

Proper axial placement of the vascular prosthesis of the invention preferably is achieved using radiopaque markers on the delivery catheter and/or the vascular prosthesis. For example, the markers may be disposed at the center or ends of the feature, thereby allowing the feature to be placed at the desired location with respect to an aneurysm neck.

With respect to FIG. 8, a preferred method of delivering the prosthesis of the present invention having feature F within vessel V now is described. Initially, prosthesis 40 is loaded onto the inner member of 62 of delivery catheter 60 having mark 50 that identifies an edge or the center of feature F. The vascular prosthesis is oriented radially so that it will open in the vessel in a known orientation. Vascular prosthesis 40 is delivered across aneurysm neck N, where helical section 42 becomes anchored against healthy tissue on either side of aneurysm A.

In accordance with the present invention, the delivery catheter preferably has an elliptical cross-section including major axis L₁ and minor axis L₂ that preferentially disposes the feature towards the outer radius of the vessel during transluminal advancement, as illustrated in FIG. 8. Once the prosthesis is properly positioned within vessel V, as determined, for example, by using fluoroscopic imaging, the prosthesis is deployed by retracting the outer sheath. Distal section 44 of the prosthesis deploys first by self-expanding into contact with healthy tissue distal to the aneurysm location, and helical section 42 then unwinds from inner member 62 into contact with the wall of the vessel turn-by-turn. Because the prosthesis does not foreshorten during deployment, feature 48 may be accurately place across neck N of the aneurysm. Following placement of prosthesis 40, a microcatheter may be advanced through the struts of the prosthesis to place embolization coils within the sac of aneurysm A.

While preferred illustrative embodiments of the invention are described above, it will be apparent to one skilled in the art that various changes and modifications may be made therein without departing from the invention. The appended claims are intended to cover all such changes and modifications that fall within the true spirit and scope of the invention.

What is claimed is:

1. A prosthesis for treating a vascular aneurysm, the prosthesis comprising:
a distal section;
a helical section coupled to the distal section at a junction, the helical section comprising a plurality of turns; and
a feature disposed on the helical section, the feature configured to retard or exclude blood flow into the aneurysm,
wherein the prosthesis has a contracted delivery configuration and an expanded deployed configuration, and adjacent turns of the helical section overlap one another in the contracted delivery configuration.

2. The prosthesis of claim 1, wherein the feature comprises an area of the helical section having a locally denser concentration of material than adjacent regions of the helical section, wherein the area is configured to span the neck of the aneurysm.

3. The prosthesis of claim 2, wherein the feature comprises a multiplicity of struts.

4. The prosthesis of claim 1, wherein the feature comprises graft material disposed on a predetermined area of the helical section.

5. The prosthesis of claim 1, wherein the feature is disposed at a predetermined axial and angular position on the helical body during manufacture.

6. The prosthesis of claim 1, further comprising a radio-opaque mark disposed on the helical section that indicates the location of the feature.

7. The prosthesis of claim 5, wherein the angular position of the feature is determined by the equation: r=360*x/Π*D*tan(θ),
wherein r is an angular distance from a predefined reference point,
x is an axial distance from the reference point,
θ is an angle of the helical section in the expanded deployed configuration and
D is a diameter of the helical section in the expanded deployed configuration.

8. The prosthesis of claim 1, wherein the angular location of the feature may be determined at different diameters from the equation: r₂=D₁*r₁/D₂,
wherein r₁ is the initial angular location,
D₁ is a diameter of the helical section in the contracted delivery configuration, and
D₂ is a diameter of the helical section in the expanded deployed configuration.

9. A method of marking a location of a feature on a prosthesis to be placed adjacent to an aneurysm using a delivery catheter, the prosthesis having a contracted delivery configuration and an expanded deployed configuration, the delivery catheter comprising a helical ledge affixed to its outer surface, the method comprising steps of:
selecting a reference point on the delivery catheter;
determining an axial location of the reference point;
determining an angular location of the feature; and
placing a radio-opaque mark on the prosthesis to indicate the expected location of the feature.

10. The method of claim 9, wherein a distal end of the helical ledge is used as the reference point.

11. The method of claim 9, wherein the axial location of the feature is predetermined, and the angular location of the feature is determined using the formula: r=360*x/Π*D*tan(θ),
wherein r is angular distance from the reference point,
x is an axial distance from the reference point,
θ is an angle of the helical section in the expanded deployed configuration and
D is a diameter of the helical section in the expanded deployed configuration.

12. The method of claim 9, further comprising placing the prosthesis adjacent to the aneurysm using the radio-opaque mark disposed on the prosthesis.

13. Apparatus for treating an aneurysm, the apparatus comprising:
a prosthesis comprising a self-expanding helical section having a contracted delivery configuration and a deployed configuration; and
a delivery catheter comprising:
a sheath having proximal and distal ends and a lumen extending therethrough; and
an inner member configured to be slidably received within the lumen of the sheath, the inner member having an outer surface defining a helical ledge and a non-circular cross-section, wherein the non-circular cross-section facilitates angular orientation of the prosthesis within a body vessel.

14. The apparatus of claim 13, wherein the inner member has an elliptical cross-section.

15. The apparatus of claim 13, wherein the inner member includes major and minor axes, the major axis configured to place the prosthesis in apposition to an aneurysm.

16. The apparatus of claim 13, wherein the prosthesis includes a feature disposed on the helical section, the feature configured to exclude or retard blood flow into the aneurysm.

17. The apparatus of claim 15, wherein the feature comprises an area of locally higher strut density.

18. The apparatus of claim 15, wherein the feature comprises an area of graft material.

19. The apparatus of claim 13, wherein the non-circular cross-section of the inner member causes the delivery catheter to enter tortuous anatomy with a known orientation.

20. The apparatus of claim 13, wherein the delivery catheter automatically orients itself within a vessel so that a feature of the prosthesis is disposed in apposition to the aneurysm.
